(19) United States
(12) Patent Application Publication
(10) Pub. No.: US 2010/0173028 A1
(43) Pub. Date: Jul. 8, 2010

(54) PROCESS FOR PREPARING VITIS VINIFERA PIP EXTRACT AND PHARMACEUTICAL COMPOSITION FOR PREVENTING OR TREATING RHEUMATOID ARTHRITIS COMPRISING THE SAME

(76) Inventors: Jun-Ki Min, Gyeonggi-do (KR); Mi-La Cho, Seoul (KR); Mi-Kyung Park, Gyeonggi-do (KR); Yu-Jung Heo, Seoul (KR); Jin-Sil Park, Seoul (KR); Ho-Youn Kim, Seoul (KR); Jong-Hyeon Ryu, Gyeonggi-do (KR); Hyun-Gyu Kim, Gyeonggi-do (KR); Geun-Hyeog Lee, Gyeonggi-do (KR)

Correspondence Address: THE NATH LAW GROUP, 112 South West Street, Alexandria, VA, 22314, US

(21) Appl. No.: 12/733,461

(22) PCT Filed: Sep. 4, 2008
(86) PCT No.: PCT/KR2008/005203

§ 371 Date: Mar. 3, 2010

(30) Foreign Application Priority Data
Sep. 4, 2007 (KR) 10-2007-0089273

(51) Int. Cl. A61K 36/87; A61K 31/353; A61P 29/00

(52) U.S. Cl. 424/766; 514/456

(57) ABSTRACT

The present invention provides an improved process for preparing a Vitis vinifera pip extract. And also, the present invention provides a pharmaceutical composition for preventing or treating rheumatoid arthritis including the Vitis vinifera pip extract as an active ingredient.

### TECHNICAL FIELD

The present invention relates to a process for preparing a Vitis vinifera pip extract and a pharmaceutical composition for preventing or treating rheumatoid arthritis including the Vitis vinifera pip extract as an active ingredient.

### BACKGROUND ART

Genus Vitis is a climbing plant belonging to order Rhamnales and family Vitaceae and naturally grows or is cultivated all over the earth except for equator areas and areas with latitude of 50° or higher. 11 genera and 700 species belong to the family Vitaceae. Vitis vinifera, Vitis labrusca, Vitis riparia, Vitis rupestris, Vitis berladieri, Vitis coignetiae, Vitis amurensis, and the like are cultivated for fruits.

An extract, which is obtained from the seeds of Vitis vinifera, includes (−)epicatechin, proanthocyanidins B1 and B2, (+)catechin, and a mixture of their polymerization derivatives, which are known as procyanidol or flavanol oligomer (GB-A-1541469 and FR-A-2092743). It is known that the extract is useful for the treatment of circulatory diseases.

U.S. Patent Publication No. 2003/0165589 discloses that Vitis vinifera, preferably an extract obtained from leaves of Vitis vinifera, inhibits Nitric Oxide-synthase (NO-synthase), and thus can be used to inhibit a variety of diseases related to NO-synthase, such as cellular differentiation and/or proliferation, growth of epidermis and/or hyperproliferative disorders, degradation and destruction of cells, and immunological and/or inflammatory processes.

Meanwhile, U.S. Patent Publication No. 2006/0280811 discloses formulations for the treatment of arthritis including saligenin or Salix rubra extract; boswellic acid or Boswellia serrata extract; procyanidins from Vitis vinifera or from Camellia sinensis, preferably complexed with phospholipids, rhein, or lipophilic derivatives thereof; N-acetyl-glucosamine; and glucronic acid or glucuronolactone. According to U.S. Patent Publication No. 2006/0280811, the procyanidins synergistically interact with cyclooxygenanse-2 (COX-2) inhibiting components present in the Salix and Boswellia extract. However, it is not disclosed what component specifically acts as active ingredients for the treatment of arthritis. The procyanidins can be obtained using an extracting method disclosed in GB-A-1541469 (and FR-A-2092743) or U.S. Pat. No. 5,484,594 (EP 348,781).

U.S. Pat. No. 4,963,527 discloses cosmetic compositions containing phospholipid complexes of Vitis vinifera extract, which can be also obtained as described in FR-A-2092743 (i.e., GB-A-1541469).

According to GB-A-1541469 (and FR-A-2092743), an acetone-water mixture containing 3 to 4 volumes of acetone to one volume of water is used as a first extraction solvent in order to extract oligomers and remove impurities by precipitation (i.e., partial precipitation). That is, Vitis vinifera (e.g., seed) is treated with the acetone-water mixture containing 3 to 4 volumes of acetone to one volume of water, and acetone is removed by distillation. Then, precipitated impurities are filtered, and the aqueous filtrate is treated with a water-immiscible organic solvent such as ethyl acetate. Then, the organic solvent is removed, and the residue is removed. The remaining impurities are removed by precipitation by adding sodium chloride, and the aqueous filtrate is treated with ethyl acetate and chloroform. According to GB-A-1541469, since partial precipitation does not occur when excessive amount of water is used, the use of excessive amount of water cannot be effectively applied to mass production.

Extraction disclosed in GB-A-1541469 should be conducted using a mixture including excessive amount of acetone as a first extraction solvent at the boiling temperature of acetone (at about 57° C.) and the excessive amount of acetone should be removed by distillation. Therefore, costs for the extraction may be increased due to heating conditions and long evaporation time, and environmental contamination may be also caused due to the use of excessive amount of acetone. In addition, acetone may remain in the finally obtained extract. Furthermore, the extraction process may be prolonged since treatment using a water-immiscible organic solvent such as ethyl acetate and removal of the ethyl acetate are necessary before adding sodium chloride.

U.S. Pat. No. 5,484,594 (European Patent No. 348,781) discloses a selective extraction method using ultrafiltration on membranes of cut-off from 3000 to 600; and using ethers, or esters or mixtures of ethyl acetate and aromatic hydrocarbons. However, costs for extraction is high since more extraction processes are necessary compared with the method disclosed in GB-A-1541469, and additional equipments, such as equipment for ultrafiltration, needs to be used.

Meanwhile, Korean Patent No. 10-0509119 discloses that a procyanidin oligomer (catechin-4-phloroglucinol), obtained from Ulmi cortex using a multi-step extraction process including column chromatography, inhibits activity of matrix metalloproteinase (MMP), and thus can be efficiently used to prevent and treat diseases such as cancer migration, periodontal diseases, rheumatoid arthritis, inflammation, hyperparathyroidism, diabetes, corneal ulcer, osteoporosis, stomach ulcer, trauma, wrinkles, acne, AIDS, burn, arteriosclerosis, bone fracture. However, it does not disclose the method of obtaining Vitis vinifera extract. Korean Patent No. 10-0509119 discloses only that procyanidin oligomers are abundant in Ulmi cortex, grape seeds, rhubarb, Pleuropterus multiflorus, camphor trees, cinnamon, Thuja orientalis, camellia, millet, buckwheet, oak, and the like, but it is not disclosed that extract of graph seeds, particularly, Vitis vinifera extract could be related to rheumatoid arthritis.

### DETAILED DESCRIPTION OF THE INVENTION

### Technical Problem

The present inventors conducted research in order to develop an improved process for preparing a Vitis vinifera extract through a simple process. As a result, the present inventors have found that, by extracting Vitis vinifera seeds using a water-acetone mixture containing a relatively small amount of acetone as a first extraction solvent, removing acetone by distillation, and then directly treating the resultant aqueous layer with sodium chloride, the extraction can be performed at room temperature (at about 25° C.) and also distillation can be simplified. In addition, it has been also found that the extraction process can be simplified since it is not necessary to perform extraction step using a water-immiscible organic solvent.

The present inventors have also found an improved process for preparing the extract, the process including separately preparing a first extract and a second extract, and mixing them. That is, the extract can be prepared by preparing a first extract obtained by conducting extraction using an acetone-water mixture and then conducting extraction using ethyl acetate; preparing a second extract obtained by conducting extraction using only water as an extraction solvent and conducting extraction using ethanol; and mixing the first extract and the second extract. According to the process, the amount of acetone used can be decreased, extraction process can be simplified since sodium chloride saturation and chloroform extraction are not necessary, and environmental contamination caused by using organic solvents can be minimized. In particular, the yield of the extract can be increased by about 10 times.

The present inventors conducted research on various pharmacological effects of a Vitis vinifera pip extract and discovered that the Vitis vinifera pip extract has excellent effects on preventing and treating rheumatoid arthritis. It is very surprising since it has not been reported that the Vitis vinifera extract could be related to rheumatoid arthritis.

Thus, the present invention provides improved processes for preparing a Vitis vinifera pip extract.

The present invention also provides a pharmaceutical composition for preventing or treating rheumatoid arthritis comprising a Vitis vinifera pip extract as an active ingredient.

### Technical Solution

According to an aspect of the present invention, there is provided a process for preparing a Vitis vinifera pip extract, the process comprising: (a) extracting pulverized seeds of Vitis vinifera at room temperature with a mixed solvent of acetone and water in a volume ratio of 1:1-2, and then filtering the resultant; (b) distilling the filtrate prepared in step (a) to remove acetone, saturating with sodium chloride, and then filtering the resultant; (c) extracting the filtrate prepared in step (b) with ethyl acetate, and then concentrating the resultant; and (d) adding chloroform to the concentrate prepared in step (c), and then obtaining the resultant precipitate by filtration.

The volume ratio of acetone and water in the mixed solvent of step (a) may be 1:1.5. The extraction of steps (a) and (c) may be repeated 2 to 3 times. The distillation of step (b) may be conducted at 50° C. or less under a reduced pressure and the filtering of step (b) may be conducted after standing for 2 to 3 hours from the saturation with sodium chloride. The concentrating in step (c) may be conducted to a volume of 0.4 to 0.7 times of the total volume of the extract.

According to another aspect of the present invention, there is provided a process for preparing a Vitis vinifera pip extract, the process comprising: (A) preparing a first extract by (i) extracting pulverized seeds of Vitis vinifera with a mixed solvent of acetone and water in a volume ratio of 3-5:1, and then filtering the resultant; (ii) concentrating the filtrate prepared in step (i) to remove acetone, and then filtering the resultant; (iii) extracting the filtrate prepared in step (ii) with ethyl acetate; and (iv) drying the extract prepared in step (iii); (B) preparing a second extract by (p) extracting pulverized seeds of Vitis vinifera with water, and the filtering the resultant; (q) extracting the filtrate prepared in step (p) with ethanol, and then filtering the resultant; and (r) drying the filtrate prepared in step (q); and (C) mixing the first extract and the second extract.

The volume ratio of acetone and water in the mixed solvent of step (i) may be 4:1. The extraction of step (iii) or (q) may be repeated 2 to 3 times. Step (iii) may further comprise dehydrating the resulting extract. The drying of step (iv) may be conducted by concentrating the extract prepared in step (iii) to remove ethyl acetate, dissolving the concentrate in water, and then spray-drying the resultant. The drying of step (r) may be conducted by spray-drying the filtrate prepared in step (q) or by concentrating the filtrate prepared in step (q), and then spray-drying the concentrate. The mixing ratio of the first extract and the second extract may be 1:0.5-1.5 by weight.

It is preferable that the obtained Vitis vinifera pip extract in the above processes have 80-130% of Procyanidolic value (PCV); 30% or less of (+)catechin and (−)epicatechin; and 95-105% of proanthocyanidin.

According to still another aspect of the present invention, there is provided a pharmaceutical composition for preventing or treating rheumatoid arthritis comprising a Vitis vinifera pip extract as an active ingredient and a pharmaceutically acceptable carrier. Preferably, the Vitis vinifera pip extract is prepared in accordance with the above processes. More preferably, the Vitis vinifera pip extract has 80-130% of Procyanidolic value (PCV); 30% or less of (+)catechin and (−)epicatechin; and 95-105% of proanthocyanidin.

### ADVANTAGEOUS EFFECTS

According to the present invention, extraction is conducted using a water-acetone mixture having relatively small amount of acetone as a first extraction solvent. Therefore, acetone can be simply distilled and residual solvent can be minimized. Furthermore, a heating process is not necessary since the extraction can be performed at room temperature (at about 25° C.), and extraction using a water-immiscible organic solvent is not necessary before adding sodium chloride. Thus, the process for preparing a Vitis vinifera pip extract according to the present invention can be conducted simply and cost-effectively, and thus can be suitably applied to industrial mass production.

In addition, according to a process for preparing a Vitis vinifera pip extract by separately preparing a first extract and a second extract and mixing them, the amount of acetone can be decreased, extraction process can be simplified since sodium chloride saturation and chloroform extraction are not necessary, and environmental contamination caused by using organic solvents can be minimized. In particular, the yield of the extract can be increased by about 10 times.

Furthermore, the Vitis vinifera pip extract prepared according to the process, as a natural extract, has excellent safety and shows excellent preventing and treating effects of rheumatoid arthritis, e.g., in a collagen induced arthritis (CIA) animal model.

### BRIEF DESCRIPTION OF THE DRAWINGS

FIG. 1 is a graph illustrating arthritis scores of collagen induced arthritis (CIA) animals, CIA animals into which 100 mg/kg of Vitis vinifera pip extract (GSPE) is injected in 5 consecutive intraperitoneal injection, and CIA animals into which saline is injected 5 consecutive intraperitoneal injection, observed for 10 weeks.

FIG. 2 illustrates photographs of joint regions of CIA animals, CIA animals into which the Vitis vinifera pip extract is injected, and normal animals before sacrificing them.

FIG. 3 illustrates the degree of destruction of joints and cartilaginous tissues of CIA animals, CIA animals into which each of 50 mg/kg and 10 mg/kg of the Vitis vinifera pip extract is injected, and normal animals after sacrificing them, through tissue staining.

FIG. 4 illustrates the amount of Type II collagen-specific antibodies (anti-CII IgG1 and anti-CII IgG2a) in serums of CIA animals into which 100 mg/kg of the Vitis vinifera pip extract is injected, CIA animals into which saline is injected, and normal animals.

FIG. 5 illustrates the amount of IL-17 and TNF-α measured by enzyme-linked immunosorbent assay (ELISA), in a culture supernatant prepared by co-culturing thymus CD4+ T cells and spleen CD11c+ dendritic cells in a ratio of 10:1 for 3 days, which are obtained from CIA animals into which 100 mg/kg of the Vitis vinifera pip extract is injected or CIA animals into which saline is injected.

FIG. 6 illustrates the amount of IL-17 and IL-4 in a culture supernatant prepared by co-culturing the cells obtained in the same manner as described in FIG. 5 for 3 days in culture media unsupplemented or supplemented with 10 μg/ml and 20 μg/ml of the Vitis vinifera pip extract with stimulation of anti-CD3 using ELISA.

FIG. 7 illustrates the degree of expression of IL-17 mRNA, using a real-time transcriptase-polymerase chain reaction (PCR), from draining lymph nodes (dLN) which are obtained from CIA animals into which 50 mg/kg and 10 mg/kg of Vitis vinifera pip extract or saline are respectively injected, and cultured in culture media unsupplemented or supplemented with 10 μg/ml of the Vitis vinifera pip extract with stimulation of anti-CD3, CII, and lipopolysaccharide (LPS) for 3 days.

FIGS. 8 and 9 illustrate the results of co-culturing thymus CD4+ T cells and spleen CD11c+ dendritic cells obtained from a CIA animal model and an autoimmune arthritis animal model IL-1Ra−/− in a ratio of 10:1 in culture media unsupplemented or supplemented with 10 μg/ml of the Vitis vinifera pip extract with stimulation of anti-CD3, CPG or CII, for 6 days, using a fluorescence activated cell sorter (FACS).

FIG. 10 is a graph illustrating arthritis score of CIA animals into which saline, celecoxib, or 10% DMSO are injected via intraperitoneal injection, or the Vitis vinifera pip extract is orally administered.

FIG. 11 illustrates a joint piece of CIA animals, dyed with hematoxylin and eosin, into which saline, celecoxib, or 10% DMSO are injected via intraperitoneal injection, or the Vitis vinifera pip extract is orally administered.

FIGS. 12 and 13 illustrate subtypes of Total IgG antibody of CIA animals into which saline is injected via intraperitoneal injection or the Vitis vinifera pip extract is orally administered.

FIGS. 14 to 16 illustrate the amount of IL-17, TNF alpha, and IL-1β in CIA animals into which saline, celecoxib, or 10% DMSO are injected via intraperitoneal injection or the Vitis vinifera pip extract is orally administered.

### BEST MODE FOR CARRYING OUT THE INVENTION

The present invention includes a process for preparing a Vitis vinifera pip extract, the process comprising: (a) extracting pulverized seeds of Vitis vinifera at room temperature with a mixed solvent of acetone and water in a volume ratio of 1:1-2, and then filtering the resultant; (b) distilling the filtrate prepared in step (a) to remove acetone, saturating with sodium chloride, and then filtering the resultant; (c) extracting the filtrate prepared in step (b) with ethyl acetate, and then concentrating the resultant; and (d) adding chloroform to the concentrate prepared in step (c), and then obtaining the resultant precipitate by filtration.

Pulverized seeds of Vitis vinifera may be prepared by compressing Vitis vinifera to obtain bark, seed, and branch, washing the resultants with water, drying the same using an oven, isolating the seeds, and then pulverizing the seeds using a conventional method.

In the process of the present invention, a mixed solvent of acetone and water having a lower amount of acetone compared with that used in a conventional extraction (e.g., disclosed in GB-A-1541469) is used as a first extraction solvent and the first extraction process is conducted at room temperature (at about 25° C.) without heating. In the mixed solvent of acetone and water used herein, the volume ratio of acetone to water may be in the range of 1:1-2, and preferably about 1:1.5. The first extraction may be conducted once or repeated several times, preferably 2 to 3 times. The filtration of step (a) may be conducted using a conventional method, and the filtrate is collected for the next step.

The process of the present invention includes distilling the filtrate prepared in step (a) to remove acetone, saturating with sodium chloride, and then filtering the resultant [step (b)]. During the distillation, acetone having a relatively low boiling point is removed, and thus impurities dissolved in acetone are precipitated. The distillation may be performed using a conventional method, for example, under a reduced pressure. The distillation may be performed at about 50° C. or less under a reduced pressure. The extract obtained using the distillation is not subjected to extraction using an organic solvent, but saturated with sodium chloride and then filtered. If the extract (i.e., extract in which acetone is evaporated) is saturated with sodium chloride, impurities such as tannin are precipitated and removed during the filtration. In the precipitation of impurities through the saturation with sodium chloride and filtration, the filtration may be conducted after standing for 2 to 3 hours from the saturation with sodium chloride. The filtration may be conducted using a conventional method, and the filtrate is collected for the next step.

The process of the present invention includes extracting the filtrate prepared in step (b) with ethyl acetate, and then concentrating the resultant [step (c)]. The extraction using ethyl acetate (second extraction) may be conducted once or repeated several times, preferably 2 to 3 times. The concentrating may be conducted to a volume of 0.4 to 0.7 times of the total volume of the obtained extract.

The process of the present invention includes adding chloroform to the concentrate prepared in step (c), and then obtaining the resultant precipitate by filtration [step (d)]. When chloroform is added to the concentrate, active ingredients including oligomers which are not dissolved in chloroform are precipitated. The Vitis vinifera pip extract may be simply isolated by filtering the precipitate. The precipitate prepared by the filtration may be dried using a conventional method to obtain dried powder. The drying may be conducted under a reduced pressure, for example, at 50° C. or less under a reduced pressure.

The present invention also includes a process for preparing a Vitis vinifera pip extract, the process comprising: (A) preparing a first extract by (i) extracting pulverized seeds of Vitis vinifera with a mixed solvent of acetone and water in a volume ratio of 3-5:1, and then filtering the resultant; (ii) concentrating the filtrate prepared in step (i) to remove acetone, and then filtering the resultant; (iii) extracting the filtrate prepared in step (ii) with ethyl acetate; and (iv) drying the extract prepared in step (iii); (B) preparing a second extract by (p) extracting pulverized seeds of Vitis vinifera with water, and the filtering the resultant; (q) extracting the filtrate prepared in step (p) with ethanol, and then filtering the resultant; and (r) drying the filtrate prepared in step (q); and (C) mixing the first extract and the second extract.

Pulverized Vitis vinifera seeds may be prepared as described above. The extract may be prepared by separately preparing a first extract and a second extract and mixing them. Thus, according to the process of the present invention, the amount of acetone may be decreased. And also, since sodium chloride saturation and chloroform extraction are not necessary, the process may be simplified and environmental contamination that may be caused by organic solvents may be minimized. In addition, the yield of the extract may be increased by about 10 times.

In the preparation of the first extract, step (i) is conducted by extracting pulverized seeds of Vitis vinifera with a mixed solvent of acetone and water in a volume ratio of 3-5:1, more preferably 4:1, and then filtering the resultant. The extraction may be conducted once or repeated several times, preferably 2 to 3 times. The filtration of step (i) may be conducted using a conventional method, and the filtrate is collected for the next step.

In the preparation of the first extract, step (ii) is conducted by concentrating the filtrate prepared in step (i) to remove acetone, and then filtering the resultant. During the concentration, acetone having a relatively low boiling point is removed, and thus impurities dissolved in acetone are precipitated. The concentration may be conducted using a conventional concentration under a reduced pressure (or distillation under a reduced pressure), for example, using distillation under the condition of a reduced pressure. The precipitate is removed by filtration, and the filtrate is collected.

In the preparation of the first extract, step (iii) is conducted by extracting the filtrate prepared in step (ii) with ethyl acetate. The extraction using ethyl acetate (second extraction) may be conducted once or repeated several times, preferably 2 to 3 times. In addition, step (iii) may further include a dehydration process, e.g., using anhydrous sodium sulfate, after the extraction using ethyl acetate.

In the preparation of the first extract, step (iv) is conducted by drying the extract prepared in step (iii). The drying may be conducted using a conventional method, for example, at 50° C. or less under a reduced pressure. More preferably, the drying may be conducted by concentrating the extract prepared in step (iii) to remove ethyl acetate, dissolving the concentrate in water, and then spray-drying the resultant.

In the preparation of the second extract, step (p) is conducted by extracting pulverized seeds of Vitis vinifera with water, and the filtering the resultant; and step (q) is conducted by extracting the filtrate prepared in step (p) with ethanol, and then filtering the resultant. The extraction of step (q) may be conduced once or repeated several times, preferably 2 to 3 times.

In the preparation of the second extract, step (r) is conducted by drying the filtrate prepared in step (q). The drying of step (r) may be conducted by spray-drying the filtrate prepared in step (q); or concentrating the filtrate of step (q) and then spray-drying the concentrate. The concentration may be conducted into a volume of 0.4 to 0.7 times of the total volume of the filtrate prepared in step (q), but is not limited thereto.

The mixing the first extract and the second extract may be conducted by simply mixing the extracts. The mixing ratio of the first extract and the second extract may be 1:0.5-1.5 by weight.

The Vitis vinifera pip extract prepared according to the process of the present invention has 80-130% of Procyanidolic value (PCV); 30% or less of (+)catechin and (−)epicatechin; and 95-105% of proanthocyanidin.

The “procyanidolic value (PCV)” used herein is calculated as follows.

\textcircled{1} Preparation of Reference Solution

100 mg of Vitis vinifera pip extract standard is precisely measured and mixed with isopropanol to form a solution having a volume of 50 ml. 10 ml of the solution is mixed with 10 ml of 3M hydrochloric acid, and then the obtained solution is mixed with isopropanol to prepare a reference solution having a volume of 50 ml.

\textcircled{2} Preparation of Test Solutions

100 mg of each test samples are processed in the same manner of preparation of the standard solution to prepare test solutions.

\textcircled{3} Measurement

10 ml of each of the test solutions and the reference solutions are respectively added to 5 test tubes and the test tubes are capped. Then, the test tubes are heated in a water bath at 100° C. for 45 minutes. After heating, the test tubes are cooled in cold water, and 2 ml of the solution obtained from each of the test tubes is mixed with 20 ml of isopropanol. Absorbance is measured at 550 nm for the test solution and the reference solution, using isopropanol as a control solution, and 5 average absorbance values were calculated.

\textcircled{4} Calculation

PCV=105×[A(t)×Mt×(100−Et)]/[A(s)×M×(100−E)]
A(t): mean absorbance of the test solutions
A(s): mean absorbance of the reference solutions
Mt: mass in mg of the standard
M: mass in mg of the test sample
Et: water content of the standard (%)
E: water content of the test sample (%)

The (+)catechin and (−)epicatechin are quantified as follows.

\textcircled{1} Preparation of Reference Solution

50 mg of each of (+)catechin standard and (−)epicatechin standard are dissolved in a mixture of acetonitrile and diluted phosphoric acid (5:95) to prepare a reference solution having a volume of 100 ml.

\textcircled{2} Preparation of Test Solutions

50 mg of each test samples are dissolved in a mixture of acetonitrile and diluted phosphoric acid (5:95) to prepare a test solution having a volume of 10 ml.

\textcircled{3} Assay Condition
Column: a column packed with octadecylsilylated silica gel (0.46×25 cm, 5 um)
Mobile phase

TABLE 1

| Time (minutes) | Mobile phase A | Mobile phase B |
|---|---|---|
| 0 | 95 | 5 |
| 50 | 85 | 15 |
| 60 | 20 | 80 |
| 70 | 95 | 5 |

Mobile phase A: diluted phosphoric acid solution (0.3%, v/v)
Mobile phase B: acetonitrile
Flow rate: 0.7 ml/min
Detector: UV detector (wavelength: 278 nm)
Injection volume: 10 ul

\textcircled{4} Calculation

Content of catechine, expressed as epicatechine (%)=[A1×Me×100×100]/[Ae×M1×(100−E)×10]

Content of epicatechine (%)=[A2×Me×100×100]/[Ae×M1×(100−E)×10]
A1: peak area of catechine in the test solution
A2: peak area of epicatechine in the test solution
Ae: peak area of epicatechine in the reference solution
Me: mass in mg of epicatechine in the reference solution
M1: mass in mg of the extract in the test solution
E: water content in the extract (%)

In addition, the “amount of proanthocyanidin” is calculated using the method described below.

\textcircled{1} Preparation of Internal Standard Solution

30.0 mg of 2,6-di-tert-butyl-4-methylphenol (BHT) is added to a 100 ml flask and mobile phase is added thereto up to the marked line to prepare an internal standard solution.

\textcircled{2} Preparation of Test Solution 1

10 mg of a sample is added to a 10 ml flask and the then dissolved in 5 ml of the internal standard solution, and the internal standard solution is added thereto up to the marked line to prepare a detection solution 1.

\textcircled{3} Preparation of Test Solution 2

10 mg of a sample is added to a 10 ml flask and the then dissolved in 5 ml of the internal standard solution, and the internal standard solution is added thereto up to the marked line to prepare a detection solution 2.

\textcircled{4} Preparation of Calibration Curve of Reference Solution
Reference solution 1: 8 mg of proanthocyanidin standard is added to a 10 ml flask and dissolved in 5 ml of the internal standard solution, and the internal standard solution is added thereto up to the marked line to prepare reference solution 1.
Reference solution 2: 10 mg of proanthocyanidin standard is added to a 10 ml flask and dissolved in 5 ml of the internal standard solution, and the internal standard solution is added thereto up to the marked line to prepare reference solution 2.
Reference solution 3: 12 mg of proanthocyanidin standard is added to a 10 ml flask and dissolved in 5 ml of the internal standard solution, and the internal standard solution is added thereto up to the marked line to prepare reference solution 3.

\textcircled{5} Assay Condition
Column: PL Gel Column (7.6×300 mm, 5 um) or similar column
Detector: UV detector (wavelength: 280 nm)
Mobile phase: mixture of tetrahydrofuran and lithium bromide solution (95:5)
   lithium bromide solution: 1.04 g of lithium bromide is added to a 1000 ml flask and water is added thereto up to the marked line.
Flow rate: 1.0 ml/min
Injection volume: 10 uL
Run time: 15 minutes

\textcircled{6} Method

Reference solutions 1, 2, and 3, and test solutions 1 and 2 are analyzed twice using liquid chromatography described below. Calibration curve of the reference solution are set using the concentration of the reference solutions and a ratio of main peak and IS peak (A_{proanthocyanidin}/A_{BHT})_{, and calculated as follows.}

\textcircled{7} Calculation

Ti%={[(A_{proanthocyanidin}/A_{BHT})ₜₑₛₜ−a]/b}×(1/Cₜₑₛₜ)×100
A_{proanthocyanidin}=peak area of proanthocyanidins in the test solution
A_{BHT}=peak area of BHT in the test solution
a=intercept of the Calibration Curve of reference solution
b=slope of the Calibration Curve of reference solution
Cₜₑₛₜ=concentration of the test solution (mg/ml)

Content of proanthocyanidins (%)=Ti%×[(100−KFstd)/(100−KFtest)]
KFstd=water content in the reference substance (%)
KFtest=water content in the test sample (%)

The present invention includes a pharmaceutical composition for preventing or treating rheumatoid arthritis comprising a Vitis vinifera pip extract as an active ingredient and a pharmaceutically acceptable carrier.

The Vitis vinifera pip extract may be prepared in accordance with the above processes. More preferably, the Vitis vinifera pip extract has 80-130% of Procyanidolic value (PCV); 30% or less of (+)catechin and (−)epicatechin; and 95-105% of proanthocyanidin.

As described in Experimental Examples below, the Vitis vinifera pip extract prepared according to the present invention shows excellent preventing and treating effects of rheumatoid arthritis, e.g., in a collagen induced arthritis (CIA) animal model.

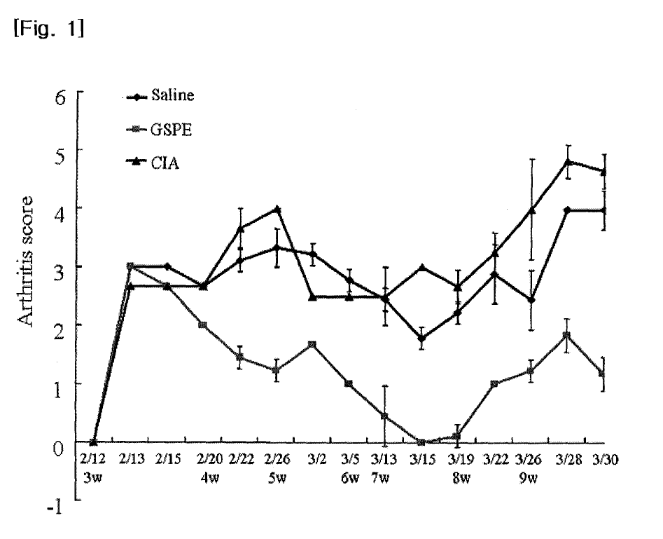

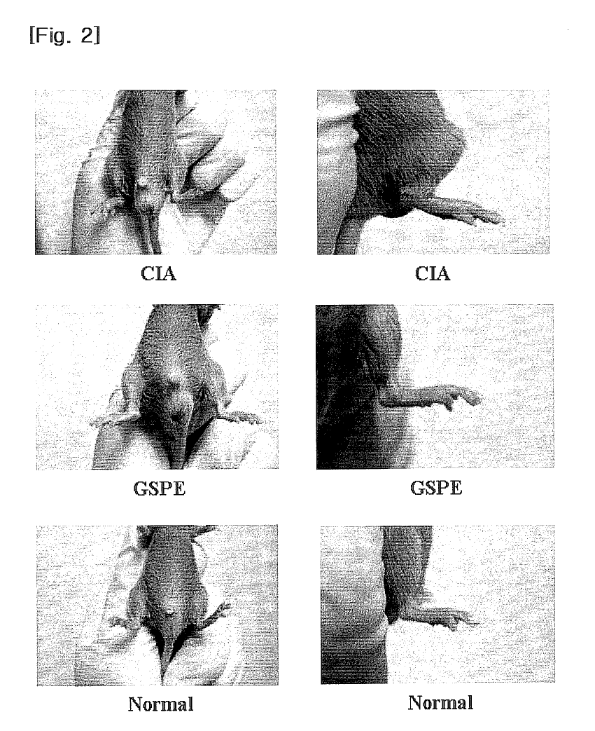

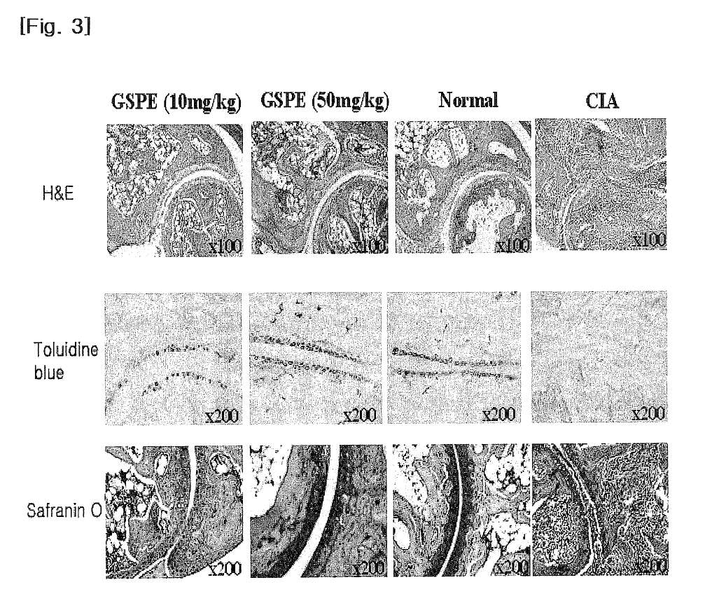

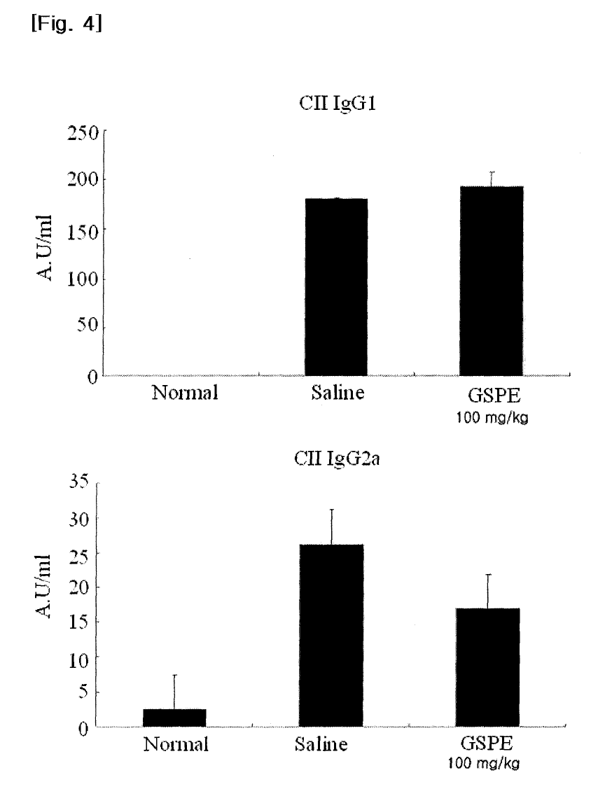

That is, when 100 mg/kg of the Vitis vinifera pip extract was injected into a CIA animal via intraperitoneal injection, arthritis score was significantly decreased (FIGS. 1 and 2). Furthermore, the relief of arthritis was observed in a rheumatoid arthritis animal model (IL-Ra−/−) having chronic inflammatory arthritis. As a result of histological assay, when 100 mg/kg of the Vitis vinifera pip extract was injected, the degree of joint destruction of the animal was similar to that of a normal mouse, and destruction of cartilage was improved (FIG. 3). In addition, as a result of serologic tests, when 100 mg/kg of the Vitis vinifera pip extract was injected, the level of Th2 type anti-CII IgG1 was not changed, but the production of CII-specific Th1 type IgG2a is decreased (FIG. 4). It can be seen that rheumatoid arthritis can be efficiently treated by administering the Vitis vinifera pip extract since the production of CII-specific Th1 type IgG2a is decreased.

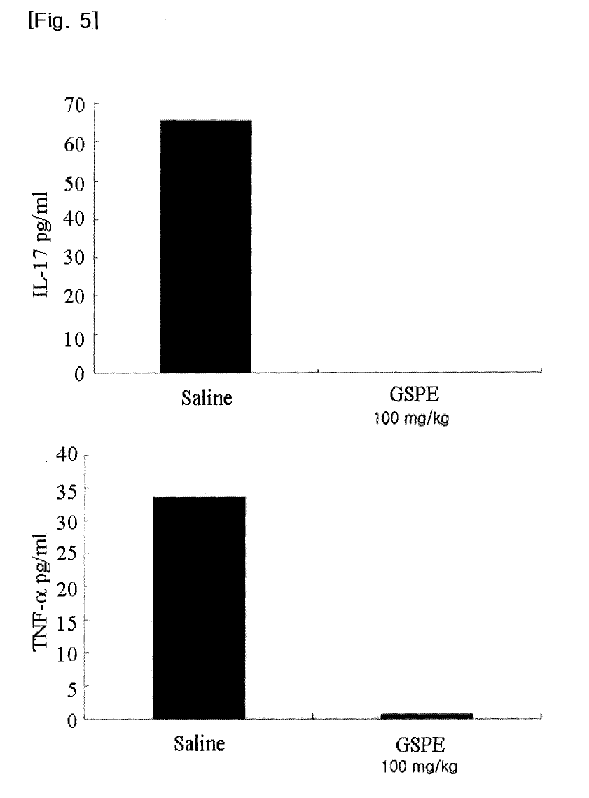

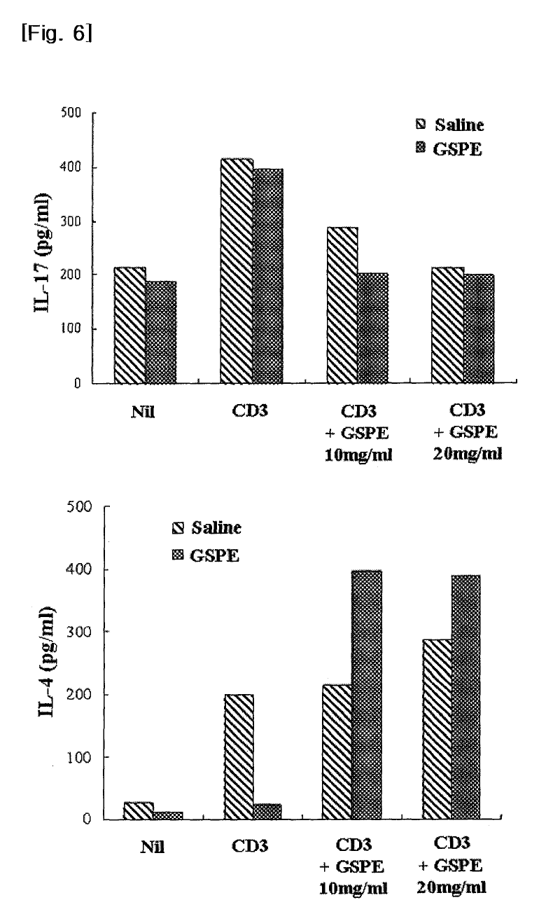

In addition, the amount of IL-17 and TNF alpha, known as inflammatory cytokines of rheumatoid arthritis, was significantly decreased when the Vitis vinifera pip extract was injected (FIG. 5), and an inflammatory cytokine IL-17 showed reverse relationship with anti-inflammatory cytokine IL-4 when Vitis vinifera pip extract was treated in vitro (FIG. 6). The profile of IL-17 at transcription level was also observed in the same pattern. When stimulating with CII or LPS and then treating with Vitis vinifera pip extract in vitro, the expression level of mRNA of IL-17 increased by CII or LPS was decreased (FIG. 7).

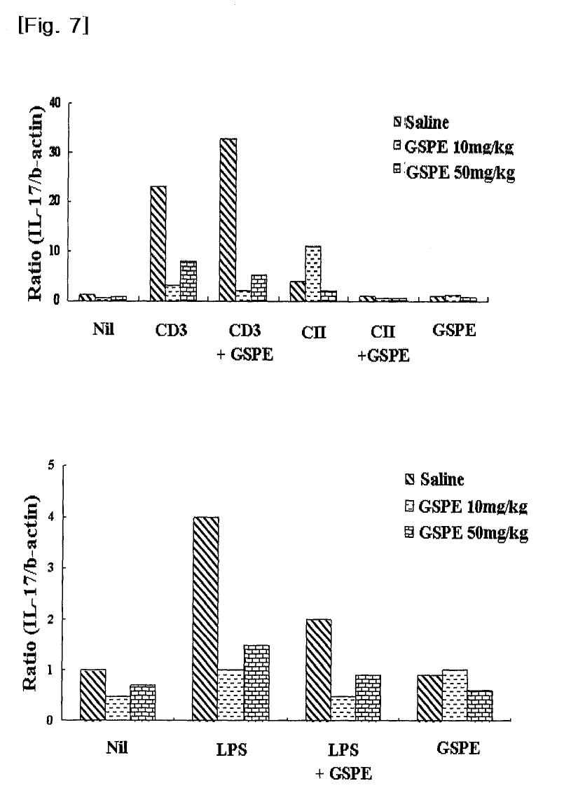

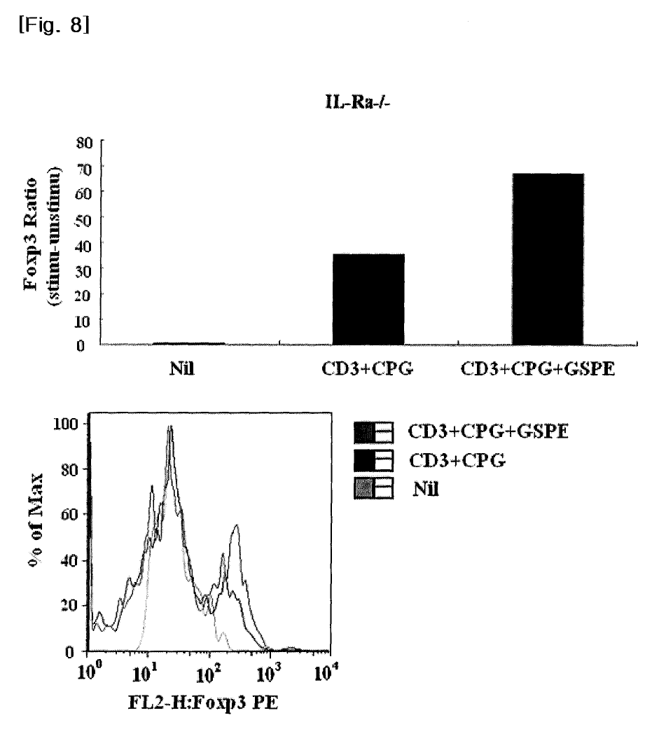

Since rheumatoid arthritis is related to excessive activity of immune cells (particularly, chronic inflammatory T cells), the regulation of CD4 T cells may be a target for the treatment. When CIA animals and IL-1Ra−/− animals were treated with a Vitis vinifera pip extract, induction of Foxp3+ regulatory CD4 T cells was increased. When CD4 T cell in CIA animals were treated with CII and Vitis vinifera pip extract, induction of Foxp3+ regulatory CD4 T cells were increased compared with the group treated only with CII (FIGS. 8 and 9). We expect that proliferation of chronic inflammatory T cells may be efficiently inhibited through generation of CII-specific regulatory CD4 T cells by the Vitis vinifera pip extract.

Thus, it can be seen that a Vitis vinifera pip extract has excellent preventing and treating rheumatoid arthritis by inhibiting generation of inflammatory cytokines and inducing anti-inflammatory cytokines and regulatory T cells.

The pharmaceutical composition of the present invention includes a pharmaceutically acceptable carrier, and can be formulated according to conventional methods into oral dosage forms such as powders, granules, tablets, capsules, suspensions, emulsions, syrups, or aerosols; external dosage forms; susppository; or sterile injection solution. Preferably, the pharmaceutical composition of the present invention may be formulated into a tablet form. The pharmaceutically acceptable carrier can be lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, or mineral oil. The pharmaceutical composition may further include a dilluent or an excipient, such as filler, expander, binder, humectant, disintegrant, or surfactant. A solid oral formulation can be a tablet, a pill, a powder, a granule, or a capsule. Such solid formulations may include at least one excipient selected from, for example, starch, calcium carbonate, sucrose, lactose, and gelatin. In addition, such solid formulations may further include a lubricant, such as magnesium stearate or talc. A liquid oral formulation can be a suspension, a solution, an emulsion, or syrup. In addition, the liquid oral formulation may include a dilluent, such as water, liquid paraffine; humectant; sweetening agent; odorant; or preservative. A parenteral formulation can be a sterile aqueous solution, a non-aqueous solution, a suspension, an emulsion, a lyophilized formulation, or a suppository. Non-aqueous solvents or suspending agents can be propylene glycol, polyethylene glycol, natural oil, such as olive oil, or injectable esters, such as ethylolate. Vehicles for suppository can be witepsol, macrogol, Tween 61, cacao butter, Laurin, or glycerogelatine.

In the pharmaceutical composition according to the present invention, a dose of the Vitis vinifera pip extract may vary depending on patient's state or body weight, seriousness of disease, dosage forms, administration routes, and the period of administration, and can be appropriately determined by a person having ordinary skill in the art. For example, the Vitis vinifera pip extract can be administered in an amount of 1 to 100 mg/kg, preferably 5 to 50 mg/kg, more preferably 5 to 10 mg/kg, per day. The administration can be completed once or through several times per day. The pharmaceutical composition of the present invention can be also administered in combination with other therapeutic agent(s) for rheumatoid arthritis. When administered as a combination, the therapeutic agent(s) can be administered sequentially or at the same time. When administered as a combination, each dose may be determined to minimum amounts showing maximum therapeutic effect, and can be appropriately determined by a person having ordinary skill in the art.

The pharmaceutical composition of the present invention can be administered to mammals, such as rats, mouse, livestock, or human beings, through various routs, e.g., orally, rectally, intravenously, intramuscularly, subcutaneously, preferably orally.

Hereinafter, the present invention will be described more specifically by the following working examples. However, the following working examples are provided only for illustrations and thus the present invention is not limited to or by them.

### Example 1

### Preparation of Vitis vinifera Pip Extract

Bark, seeds, branch of Vitis vinifera prepared by compressing Vitis vinifera were washed with water and dried in a rotating oven, and seeds of Vitis vinifera were isolated. 1 kg of the seeds were pulverized and subjected to extraction at room temperature by adding 500 ml of the mixed solution of purified water (300 ml) and acetone (200 ml). The extraction was repeated three times, and the extracts were gathered and filtered. The filtrate was distilled at 50° C. or less under a reduced pressure to remove acetone, and the resultant was saturated with sodium chloride. The resultant was placed at room temperature for 3 hours and then filtered. The filtrate was subjected to extraction three times using 250 ml of ethyl acetate, and dehydrated using anhydrous sodium sulfate. The extract was concentrated until the volume of the extract reached about 125 ml. About 600 ml of chloroform was added to the concentrate to form a precipitate, and the resultant was filtered. The precipitate was dried at 50° C. or less in a vacuum oven to obtain about 3.5 g of brown Vitis vinifera pip extract powder. The obtained extract was hydrolyzed by heating in a diluted acid solution, and PCV was measured by quantifying procyanidolic oligomers. As a result, the PCV was about 105. In addition, when measured as described in the above, the amount of proanthocyanidin was 103%. Thus, the extract includes a large amount of oligomers in which at least two monomers such as (+)catechin and (−)epicatechin were polymerized.

### Example 2

### Preparation of Vitis vinifera Pip Extract

Bark, seeds, branch of Vitis vinifera prepared by compressing Vitis vinifera were washed with water and dried in a rotating oven, and seeds of Vitis vinifera were isolated. 1 kg of the seeds of Vitis vinifera was pulverized and subjected to extraction using 500 ml of an acetone water solution (acetone/water=8/2, v/v). The extraction was repeated three times, and the extract was gathered and then filtered. The filtrate was concentrated under a reduced pressure to remove acetone and then filtered. The filtrate was subjected to extraction three times using 250 ml of ethyl acetate and dehydrated using anhydrous sodium sulfate. The extract was concentrated under a reduced pressure to remove ethyl acetate, the concentrate was dissolved in 500 ml of water, and the solution was spray-dried to obtain about 20 g of extract powder (first extract).

Bark, seeds, branch of Vitis vinifera prepared by compressing Vitis vinifera were washed with water and dried in a rotating oven, and seeds of Vitis vinifera were isolated. 1 kg of the seeds of Vitis vinifera was pulverized and subjected to extraction using 500 ml of purified water. The extraction was repeated three times, and the extract was gathered and then filtered. The filtrate was subjected to extraction three times using 250 ml of ethanol and then filtered. The filtrate was concentrated under a reduced pressure, and the concentrate was spay-dried to obtain about 15 g of extract powder (second extract).

The first extract and the second extract were mixed to obtain about 35 g of Vitis vinifera pip extract. The obtained extract was hydrolyzed by heating in a diluted acid solution, and PCV was measured by quantifying procyanidolic oligomers. As a result, the PCV was about 98. In addition, when measured as described in the above, the amount of proanthocyanidin was 98.5%. Thus, the extract includes a large amount of oligomers in which at least two monomers such as (+)catechin and (−)epicatechin were polymerized.

### Experimental Example 1

### Evaluation of Therapeutic Effect on Arthritis when Injected Via Intraperitoneal Injection

1. Preparation of Animal Model and Administration of the Vitis vinifera Pip Extract

Collagen induced arthritis (CIA) animal models were prepared and the Vitis vinifera pip extract prepared according to Example 1 was administered as follows.

6-7 week old male DBA-1 mice were used. Type 2 collagen (CII) was dissolved in 0.1N acetic acid solution to a concentration of 4 mg/ml and dialyzed with a dialysis buffer (50 mM Tris, 0.2N NaCl). The resultant was mixed with the same volume of Complete Freud's adjuvant (CFA, Chondrex) containing M. tuberculosis, and 100 μl (i.e., 100 μl/100 μg) of immunogen was hypodermically injected into the base of to tail of the mice (first injection).

After one week from the first injection, 200 μl of each of 100 mg/kg of the Vitis vinifera pip extract (dissolved in saline) and 200 μl of saline as a control group were injected into the mice via intraperitoneal injection. After one week (i.e., after two week from the first injection), the CII was mixed with the same volume of incomplete Freud's adjuvant (IFA, Chondrex), and 100 μl (i.e., 100 μl/100 μg) of the mixture was injected into one hind leg (second injection). After the second injection, 100 mg/kg of the Vitis vinifera pip extract and saline as a control group were injected via intraperitoneal injection every three days, four times in total.

5 mice were used for each group, and evaluation was conducted for 10 weeks. When arthritis score is significantly changed, each mouse was sacrificed and the activities of arthritis in blood, cells, and joint tissues were measured and various in vitro experiments were conducted.

2. Evaluation of Therapeutic Activity of Vitis vinifera Pip Extract on Rheumatoid Arthritis in CIA Animal

(2-1) Evaluation of Average Arthritis Score by Rosoliniec

Three observers who were not aware of the experiment evaluated seriousness of inflammation in joints three times a week from third week to tenth week since the first injection. The evaluation of arthritis was conducted by obtaining an average score obtained from three legs except for the leg into which CII/CFA was injected at the second injection and an average score obtained by the three observers, based on standard arthritis score by Rosioniec EF. The severity of arthritis was recorded as the mean arthritic index on a 0 to 4 scale.

The score and standard for evaluating arthritis are as follows:

0: no edema and intumescene

1: slight edema and rubefaction on feet or ankle joint

2: slight edema and rubefaction from ankle joint to metatarsal

3: medium edema and rubefaction from ankle joint to tarsals

4: edema and rubefaction from ankle to the whole leg

Since the highest arthritis score for a single leg is 4, the highest arthritis score of a mouse is 16.

It was observed that the arthritis score was gradually decreased in the animal into which the Vitis vinifera pip extract was injected. On the other hand, normal outbreak of arthritis was observed in the CIA animals and the CIA animals into which saline was injected as a control group. Thus, clinical symptoms of arthritis were different in the group treated with the Vitis vinifera pip extract and the group not treated with the Vitis vinifera pip extract (FIG. 1).

In order to identify arthritis score, photographs of the test groups were taken. In the same way as shown in the arthritis score of FIG. 1, swollen joints of the animal into which the Vitis vinifera pip extract was injected were cured to the degree of that of normal mouse (FIG. 2).

(2-2) Histological Test

50 mg/kg and 10 mg/kg of the Vitis vinifera pip extract were injected into the CIA animals prepared above in the same manner as described above. The mice were sacrificed after 8 weeks. A hind leg of each mouse was fixed using 10% formalin, decalcified and paraffined. A joint piece (5-7 μm) was dyed with hematoxylin and eosin. In addition, in order to identify the degree of destruction of cartilage, a histological test was conducted by dyeing with toluidine blue and safranin O.

As a result of the histological test, a number of immune cells were infiltrated in joints of the CIA animals and the CIA animals into which saline was injected, and pannus formation, destruction of cartilage, and bone infiltration were observed. On the other hand, the degree of destruction of joint and cartilage in the mice into which the Vitis vinifera pip extract was injected were similar to that of a normal mouse (FIG. 3).

(2-3) Serologic Test (Measuring Collagen-Specific Antibody)

In order to detect subtype of CII-specific IgG antibody using a serologic test, enzyme-linked immunosorbent assay (ELISA) was used. In mice, IgG1 among the subtypes of IgG antibody functions as a regulator inhibiting inflammation. On the other hand, IgG2a functions as a factor promoting and mediating inflammatory reactions. It has been reported that when arthritis is induced in DBA-1 mouse, IgG2a, which typically causes Th1 reaction, is specifically increased.

Serum of each of the test groups was diluted in a ratio 1:8000, and subtype of CII-specific serum IgG antibody was measured. As a result, Th2 type IgG1 was not changed, but Th1 type CII-specific IgG2a was decreased in the animal into which the Vitis vinifera pip extract was injected compared with the CIA animal (FIG. 4).

3. Evaluation of Regulation of Cytokine by the Vitis vinifera Pip Extract

(3-1) Measuring the Amount of IL-17 and TNF Alpha

After the animals of each group were sacrificed, thymus CD4+ T cells and spleen CD11c+ dendritic cells were isolated and co-cultured in a ratio of 10:1 for 3 days, and the amount of Th17 type cytokine IL-17 and Th1 type cytokine TNF alpha were measured in a supernatant using ELISA.

As a result, the two cytokines were significantly decreased in the animals into which the Vitis vinifera pip extract was injected when compared with the CIA animals (FIG. 5).

(3-2) Effect of Vitis vinifera Pip Extract on Generation of IL-17 and IL-4 by TCR Stimulation

Thymus CD4+ T cells and spleen CD11c+ dendritic cells obtained from the animals of each group were cultured for 3 days in culture media unsupplemented or supplemented with 10 μg/ml and 20 μg/ml of the Vitis vinifera pip extract with stimulation of anti-CD3, and IL-17 and IL-4 were measured in the supernatant using ELISA.

The production of IL-17, which is increased by TCR stimulation in the CIA animal into which saline was injected, was decreased depending on the concentration of the Vitis vinifera pip extract treated in vitro. The production of the anti-inflammatory cytokine IL-4 was increased depending on the concentration. On the other hand, the production of IL-17 in the animal into which the Vitis vinifera pip extract was injected was decreased greater than that in the CIA animal in vitro, and the production of IL-4 in the animal into which the Vitis vinifera pip extract was injected was increased greater than that in the CIA animal in vitro (FIG. 6).

As a result, in the animal into which the Vitis vinifera pip extract was injected, cells capable of producing anti-inflammatory cytokine IL-4 were dominant, but Th17 cells were decreased in vitro. This indicates that the Vitis vinifera pip extract regulates the amount of production of inflammatory cytokine and anti-inflammatory cytokine to treat arthritis. In addition, IL-4 is a cytokine capable of inducing regulatory T cells. Thus, it can be seen that the Vitis vinifera pip extract may induce regulatory T cells.

4. Induction of Regulatory CD4 T Cells and Inhibition of Th17 Cells by Vitis vinifera Pip Extract

In order to prove therapeutic mechanism of the Vitis vinifera pip extract for rheumatoid arthritis, immune systems that are induced or inhibited by the Vitis vinifera pip extract were examined.

(4-1) Change of mRNA Expression of IL-17 by Vitis vinifera Pip Extract

50 mg/kg and 10 mg/kg of the Vitis vinifera pip extract, and saline were respectively injected into CIA animals, and they were sacrificed. Draining lymph nodes (dLN) obtained therefrom were cultured in culture media unsupplemented or supplemented with 10 μg/ml of the Vitis vinifera pip extract with stimulation of anti-CD3, CII or LPS (lipopolysaccharide) for 3 days. Then, the degree of expression of mRNA of IL-17 was measured using real-time transcriptase-polymerase chain reaction (PCR).

The profile of IL-17 at transcription level was also observed in the same pattern as shown in FIG. 6. When the animal was stimulated with CII or LPS and treated with the Vitis vinifera pip extract in vitro, mRNA of IL-17, which was increased by CII or LPS, was decreased (FIG. 7).

(4-2) Induction of Regulatory CD4 T Cells by Vitis vinifera Pip Extract

Thymus CD4+ T cells and spleen CD11c+ dendritic cells obtained from a CIA animals and IL-1Ra−/− animals which are another rheumatoid arthritis animal were co-cultured in a ratio of 10:1 in culture media unsupplemented or supplemented with 10 μg/ml of the Vitis vinifera pip extract with stimulation of anti-CD3, CPG or CII for 6 days. Then, the degree of induction of cells expressing Foxp3 was measured using fluorescence activated cell sorter (FACS).

The induction of regulatory T cells was more increased by treating the CIA animals with CII and the Vitis vinifera pip extract compared with treating the CIA animals only with the Vitis vinifera pip extract. In addition, in the IL-1Ra−/− animal, when stimulated with the Vitis vinifera pip extract in vitro, induction of Foxp3+ regulatory CD4 T cells was identified (FIGS. 8 and 9).

As a result, when treated with CII and the Vitis vinifera pip extract, CII-specific regulatory cells were produced by increasing induction of CD4 T cells when compared with treating only with CII. Thus, excessive proliferation of chronic inflammatory T cells related to rheumatoid arthritis can be inhibited, and the inflammatory cytokine and anti-inflammatory cytokine are balanced so that the progress of rheumatoid arthritis can be inhibited and rheumatoid arthritis can be cured.

### Experimental Example 2

### Evaluation of Therapeutic Effect of Oral Administration on Arthritis

1. Preparation of Animal Model and Administration of Vitis vinifera Pip Extract

Collagen induced arthritis (CIA) animal models were prepared and the Vitis vinifera pip extract prepared according to Example 2 was administered as follows.

6-7 week old male DBA-1 mice were used. Type 2 collagen (CII) was dissolved in 0.1N acetic acid solution to a concentration of 4 mg/ml and dialyzed with a dialysis buffer (50 mM Tris, 0.2N NaCl). The resultant was mixed with the same volume of Complete Freud's adjuvant (CFA, Chondrex) containing M. tuberculosis, and 100 μl (i.e., 100 μl/100 μg) of immunogen was hypodermically injected into the base of tail of a mouse (first injection).

After one week from the first injection, 300 mg/kg of the Vitis vinifera pip extract (dissolved in saline) was orally administered to the mice. Separately, 200 μl of saline, 3 mg/kg of celecoxib (dissolved in 10% DMSO), and 10% DMSO were injected into the mice via intraperitoneal injection. After one week from the oral administration and injection (i.e., after two week from the first injection), the CII was mixed with the same volume of incomplete Freud's adjuvant (IFA, Chondrex), and 100 μl (i.e., 100 μl/100 μg) of the mixture was injected into one hind leg (second injection). After the second injection, 300 mg/kg of the Vitis vinifera pip extract, saline, 3 mg/kg of celecoxib, and 10% DMSO were orally administered or injected via intraperitoneal injection every three days, four times in total.

5 mice were used for each group, and evaluation was conducted for 8 weeks. When arthritis score is significantly changed, each mouse was sacrificed and the activities of arthritis in blood, cells, and joint tissues were measured and various in vitro experiments were conducted.

2. Evaluation of Therapeutic Activity of Vitis vinifera Pip Extract on Rheumatoid Arthritis in CIA Animal

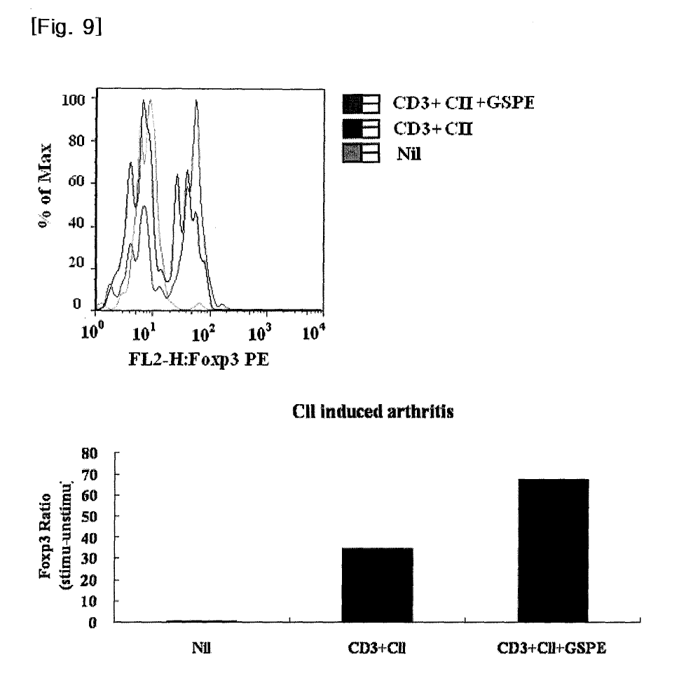

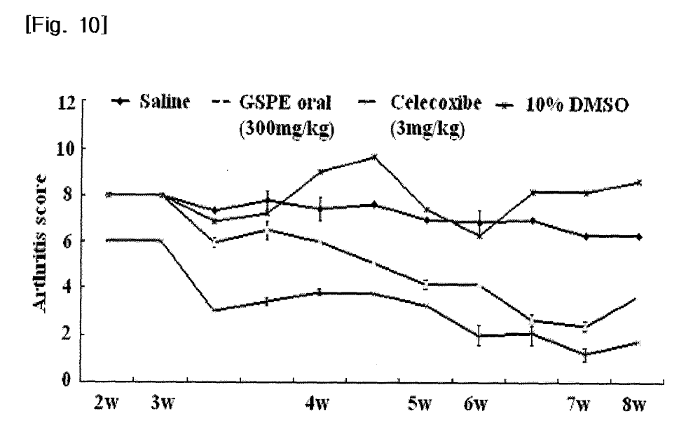

Average arthritis score was measured in the same manner as in (2-1) of Experimental Example 1, and the results are shown in FIG. 10. It was observed that the arthritis score of the animal into which the Vitis vinifera pip extract and celecoxib were administered was decreased. On the other hand, normal outbreak of arthritis was observed in the animal into which saline and 10% DMSO were injected. Clinical symptoms of arthritis were different in the group treated with the Vitis vinifera pip extract and the group not treated with the Vitis vinifera pip extract (FIG. 10).

A joint piece was dyed with hematoxylin and eosin in the same manner as in (2-2) of Experimental Example 1, and the results are shown in FIG. 11. As a result of the histological test, a number of immune cells were infiltrated in joints of the animals into which saline and 10% DMSO were injected, and pannus formation, destruction of cartilage, and bone infiltration were observed. On the other hand, the degree of destruction of joint and cartilage in the mice into which the Vitis vinifera pip extract and celecoxib were administered was similar to that of a normal mouse (FIG. 11).

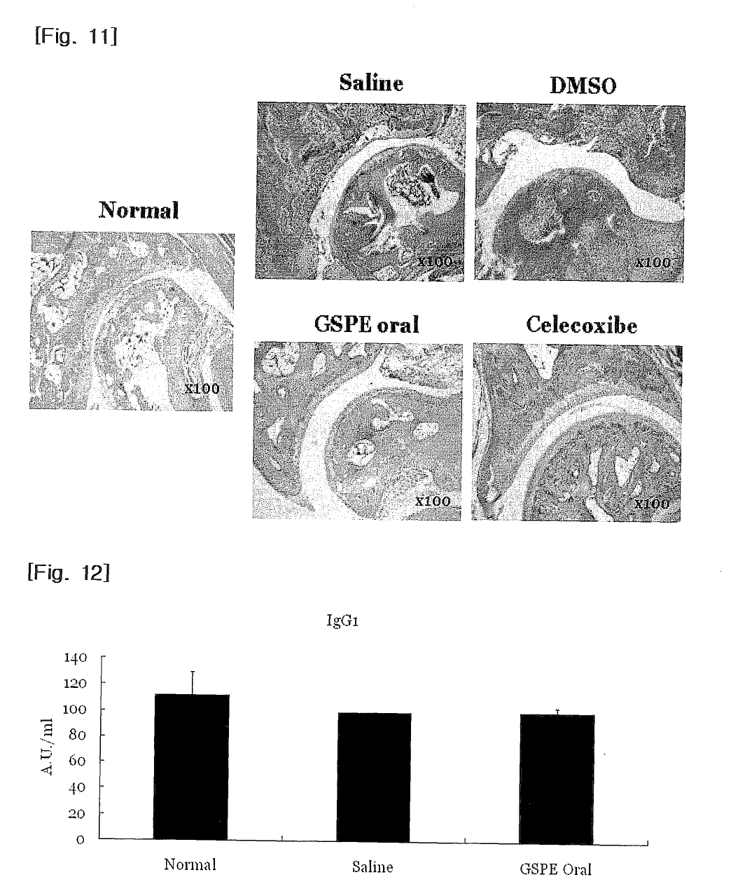

A serologic test was conducted in the same manner as in (2-3) of Experimental Example 1, and the results are shown in FIGS. 12 and 13. Serum of each of the test groups was diluted in a ratio 1:8000, and subtype of Total IgG antibody was measured. Th2 type IgG1 was not changed (FIG. 12), but Th1 type CII-specific IgG2a was decreased in the animals into which the Vitis vinifera pip extract was administered compared with the animals into which saline was injected (FIG. 13).

3. Evaluation of Regulation of Cytokine by Vitis vinifera Pip Extract

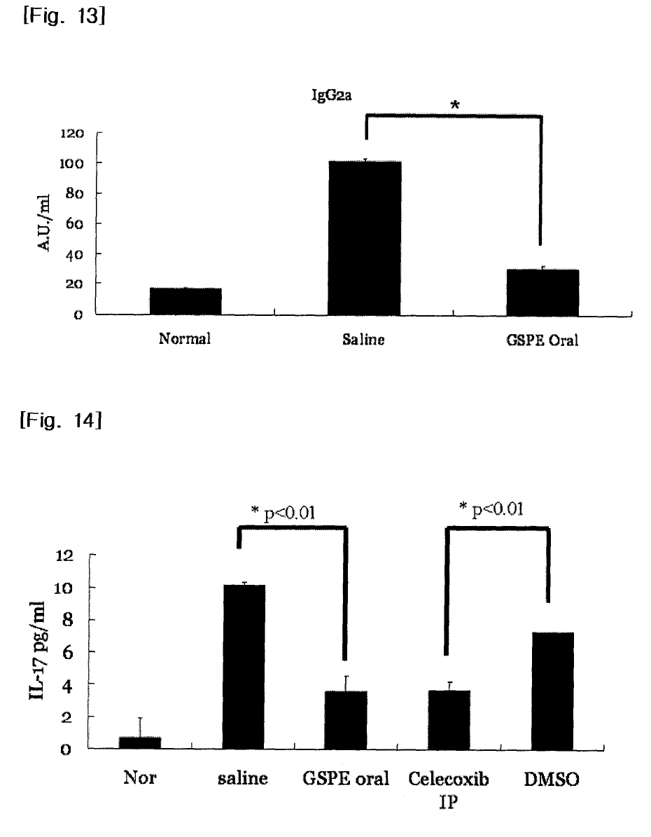

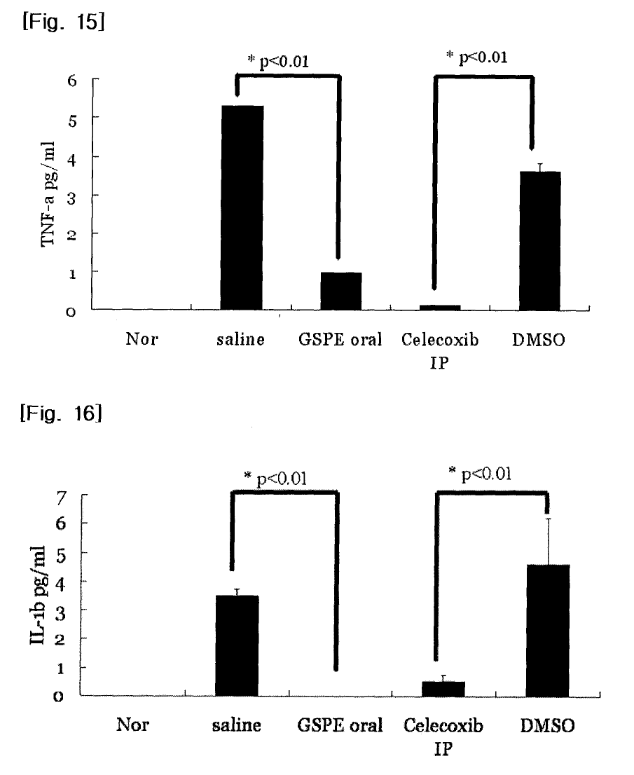

The amount of IL-17, TNF alpha, and IL-18 was measured in the same manner as in 3 of Experimental Example 1. As a result, the amount of IL-17, TNF alpha2, and IL-1β was significantly decreased in the group into which the Vitis vinifera pip extract was administered compared with the group into which saline was administered (FIGS. 14 to 16).

1. A process for preparing a Vitis vinifera pip extract, the process comprising:
(a) extracting pulverized seeds of Vitis vinifera at room temperature with a mixed solvent of acetone and water in a volume ratio of 1:1-2, and then filtering the resultant;
(b) distilling the filtrate prepared in step (a) to remove acetone, saturating with sodium chloride, and then filtering the resultant;
(c) extracting the filtrate prepared in step (b) with ethyl acetate, and then concentrating the resultant; and
(d) adding chloroform to the concentrate prepared in step (c), and then obtaining the resultant precipitate by filtration.

2. The process of claim 1, wherein the volume ratio of acetone and water in the mixed solvent of step (a) is 1:1.5.

3. The process of claim 1, wherein the extraction of steps (a) and (c) is repeated 2 to 3 times.

4. The process of claim 1, wherein the distillation of step (b) is conducted at 50° C. or less under a reduced pressure.

5. The process of claim 1, wherein the filtering of step (b) is conducted after standing for 2 to 3 hours from the saturation with sodium chloride.

6. The process of claim 1, wherein the concentrating in step (c) is conducted to a volume of 0.4 to 0.7 times of the total volume of the extract.

7. A process for preparing a Vitis vinifera pip extract, the process comprising:
(A) preparing a first extract by (i) extracting pulverized seeds of Vitis vinifera with a mixed solvent of acetone and water in a volume ratio of 3-5:1, and then filtering the resultant; (ii) concentrating the filtrate prepared in step (i) to remove acetone, and then filtering the resultant; (iii) extracting the filtrate prepared in step (ii) with ethyl acetate; and (iv) drying the extract prepared in step (iii);
(B) preparing a second extract by (p) extracting pulverized seeds of Vitis vinifera with water, and the filtering the resultant; (q) extracting the filtrate prepared in step (p) with ethanol, and then filtering the resultant; and (r) drying the filtrate prepared in step (q); and
(C) mixing the first extract and the second extract.

8. The process of claim 7, wherein the volume ratio of acetone and water in the mixed solvent of step (i) is 4:1

9. The process of claim 7, wherein the extraction of step (iii) or (q) is repeated 2 to 3 times.

10. The process of claim 7, further comprising dehydrating the resulting extract in step (iii).

11. The process of claim 7, wherein the drying of step (iv) is conducted by concentrating the extract prepared in step (iii) to remove ethyl acetate, dissolving the concentrate in water, and then spray-drying the resultant.

12. The process of claim 7, wherein the drying of step (r) is conducted by spray-drying the filtrate prepared in step (q).

13. The process of claim 7, wherein the drying of step (r) is conducted by concentrating the filtrate prepared in step (q), and then spray-drying the concentrate.

14. The process of claim 7, wherein the mixing ratio of the first extract and the second extract is 1:0.5-1.5 by weight.

15. The process according to any one of claim 1, wherein the obtained Vitis vinifera pip extract has 80-130% of Procyanidolic value (PCV); 30% or less of (+)catechin and (−)epicatechin; and 95-105% of proanthocyanidin.

16-31. (canceled)

32. A method for preventing or treating rheumatoid arthritis in a human in need thereof comprising administering an effective amount of Vitis vinifera pip extract to the human.

33. The method of claim 32, wherein the Vitis vinifera pip extract is obtained by a process comprising:
(a) extracting pulverized seeds of Vitis vinifera at room temperature with a mixed solvent of acetone and water in a volume ratio of 1:1-2, and then filtering the resultant;
(b) distilling the filtrate prepared in step (a) to remove acetone, saturating with sodium chloride, and then filtering the resultant;
(c) extracting the filtrate prepared in step (b) with ethyl acetate, and then concentrating the resultant; and
(d) adding chloroform to the concentrate prepared in step (c), and then obtaining the resultant precipitate by filtration.

34. The method of claim 32, wherein the Vitis vinifera pip extract is obtained by a process comprising:
(1) preparing a first extract by (i) extracting pulverized seeds of Vitis vinifera with a mixed solvent of acetone and water in a volume ratio of 3-5:1, and then filtering the resultant; (ii) concentrating the filtrate prepared in step (i) to remove acetone, and then filtering the resultant; (iii) extracting the filtrate prepared in step (ii) with ethyl acetate; and (iv) drying the extract prepared in step (iii);
(2) preparing a second extract by (p) extracting pulverized seeds of Vitis vinifera with water, and the filtering the resultant; (q) extracting the filtrate prepared in step (p) with ethanol, and then filtering the resultant; and (r) drying the filtrate prepared in step (q); and
(3) mixing the first extract and the second extract.

35. The method of claim 32, wherein the Vitis vinifera pip extract has 80-130% of Procyanidolic value (PCV); 30% or less of (+)catechin and (−)epicatechin; and 95-105% of proanthocyanidin.
